(19) United States
(12) Patent Application Publication
(10) Pub. No.: US 2016/0187339 A1
(43) Pub. Date: Jun. 30, 2016

(54) Biomarker and Method for Determining an Oxidative Stress Level

(71) Applicant: Biocrates Life Sciences AG, Innsbruck (AT)

(72) Inventors: Klaus Michael Weinberger, Mieming (AT); Armin Graber, Innsbruck (AT); Steven Lewis Ramsay, Viewbank (AU)

(73) Assignee: Biocrates Life Sciences AG, Innsbruck (AT)

(21) Appl. No.: 14/824,984

(22) Filed: Aug. 12, 2015

Related U.S. Application Data
(63) Continuation of application No. 13/414,581, filed on Mar. 7, 2012, now abandoned.
Application No. 13/414,581 is a continuation of application No. 12/602,472, filed on Nov. 30, 2009, now abandoned.
(60) Provisional application No. 60/924,821, filed on May 31, 2007.

(51) Int. Cl. G01N 33/573; C07D 209/16; C07D 209/20; C07C 229/36; C07C 317/48

(52) U.S. Cl. CPC G01N 33/5735; C07C 229/36; C07C 317/48; C07D 209/20; C07D 209/16; G01N 2800/7009; G01N 2333/90245

(57) ABSTRACT

The present invention relates to a biomarker and a method for determining an oxidative stress level in a biological sample, which employs co-factor-dependent oxidative stress parameters, as well as a kit adapted for carrying out such a method. In one aspect the co-factor is tetrahydrobiopterin.

### PRIORITY DATA

This application is a continuation of U.S. patent application Ser. No. 13/414,581, filed on Mar. 7, 2012, which is a continuation of U.S. patent application Ser. No. 12/602,472, filed on Nov. 30, 2009, which is a National Stage Filing of PCT Application No. PCT/EP2008/004324, filed on May 30, 2008, which claims the benefit of U.S. Provisional Patent Application Ser. No. 60/924,821, filed on May 31, 2007, each of which is incorporated herein by reference.

### technical field

This invention relates to a biomarker and a method for determining an oxidative stress level in a biological sample, which employs co-factor-dependent oxidative stress parameters, as well as a kit adapted for carrying out such a method. In one aspect the co-factor is tetrahydrobiopterin.

### BACKGROUND OF THE INVENTION

Oxidative stress measurement devices and methods have been described, for example, in WO 2006/90228, WO 2002/04029, WO 1999/63341, and EP 0 845 732.

Free radicals are atoms or molecules containing unpaired electrons. It is commonly known that these free radicals are highly reactive with membrane lipids, proteins, nucleic acids and other cellular substrates. It is also known that free radicals may be derived from environmental sources or may be generated in mammalian tissues. In order to protect these tissues as well as biological fluids from damages through free radicals, antioxidant enzymes are existing the activity of which is influenced by several co-factors.

Oxidative stress has been defined as “a disturbance in the pro-oxidant/antioxidant balance in favor of the former, leading to possible [tissue] damage” [Sies, H., Oxidative Stress. Oxidants and Antioxidants. 1991, New York: Elsevier. 507]. This balance can be related to one or more biochemical component of the biological fluid. Oxidative stress has been implicated as a key common pathway for cellular dysfunction and death and a potential therapeutic target in a broad spectrum of human medical conditions including cancer, diabetes, obstructive lung disease, inflammatory bowel disease, cardiac ischemia, glomerulonephritis, macular degeneration and various neurodegenerative disorders [Halliwell, B. and J. M. C. Gutteridge, Free Radicals in Biology and Medicine. 3 ed. 1999, Oxford: Oxford University Press Inc. 736].

Some of the end products of the cell/tissue damage, such as 3-nitrotyrosine for the nitration of proteins, 4-hydroxynonenal for the lipid peroxidation, or 8-hydroxyguanosine for nucleic acid damage, are already known, however, the detection processes are complicated and not sufficiently sensitive in order to detect gradual changes of the oxidative stress (for example, by therapeutic effects).

### SUMMARY OF THE INVENTION

It is an object underlying the present invention to provide for an improved method for determining an oxidative stress level in a biological sample which method is highly sensitive and allows for the detection of only slight changes in the oxidative stress level. Moreover, it is an object to provide for a kit being adapted for carrying out such a method.

The present invention provides a biomarker and method for determining an oxidative stress level in a biological sample which comprises measuring the activity of one ore more enzymes which enzymes depend on one or more reducing co-factors, and a kit adapted for carrying out said method. In one embodiment the co-factor is tetrahydrobiopterin (BH4). Moreover, the activity of the enzymes is determined by measuring metabolite concentrations employing a quantitative analytical method such as chromatography, spectroscopy, and mass spectrometry. It can be beneficial to use the methods and devices as described in WO 2007/003344 and WO 2007/003343 which applications are both incorporated herein by reference.

### BRIEF DESCRIPTION OF THE FIGURES

FIG. 1 describes plasma phenylalanine concentrations of diabetic mice and healthy controls (Example 1);

FIG. 2 describes plasma tyrosine concentrations of diabetic mice and healthy controls (Example 1);

FIG. 3 describes plasma phenylalanine/tyrosine ratios of diabetic mice and healthy controls (Example 1);

FIG. 4 describes increased methionine sulfoxide concentrations in high-grade (Gleason score >8) compared to low-grade (Gleason score 6) prostate tumors indicating increased oxidative stress (Example 2);

FIG. 5 describes increased tryptophan concentrations in high-grade (Gleason score >8) compared to low-grade (Gleason score 6) prostate tumors indicating impaired activity of tryptophan hydroxylase due to oxidative stress (Example 2);

FIG. 6 describes decreased serotonin concentrations in high-grade (Gleason score >8) compared to low-grade (Gleason score 6) prostate tumors indicating impaired activity of tryptophan hydroxylase due to oxidative stress (Example 2);

FIG. 7 describes increased tryptophan/serotonin ratio in high-grade (Gleason score >8) compared to low-grade (Gleason score 6) prostate tumors indicating impaired activity of tryptophan hydroxylase due to oxidative stress (Example 2);

FIG. 8 describes increasing methionine sulfoxide/methionine ratios indicating increasing oxidative stress during adaptation to high altitudes (Example 3);

FIG. 9 describes increasing phenylalanine/tyrosine ratios under increasing oxidative stress during adaptation to high altitudes indicating impaired activity of phenylalanine hydroxylase (Example 3); and

FIG. 10 describes a scheme showing a biochemical mechanism of impaired phenylalanine hydroxylase activity caused by oxidative stress.

### DETAILED DESCRIPTION OF THE INVENTION

According to the method of the present invention an enzyme activity is measured in order to determine the oxidative stress level in a biological sample. Thus, the enzyme activity is a biomarker for the oxidative stress. It is an essential feature according to the invention that the activity of the enzymes under consideration depends on the presence of at least one anti-oxidative compound. Such a compound is also referred to as a reducing co-factor.

The invention is based on the finding that an elevated level of oxidative stress, i.e. the presence of more free radicals, causes increased oxidation of certain anti-oxidative compounds which are essential co-factors for several enzymes. The lack of sufficient quantities of these co-factors then causes a reduced enzymatic activity which is determined by measuring enzyme-dependent metabolite concentrations. In particular, the ratio of substrate/product of the enzyme is determined. An increase of said ratio (decreased enzyme activity) corresponds to an increased oxidative stress level and is, thus, a parameter potentially indicative for several diseases caused by an increased oxidative stress level.

In one embodiment the reducing co-factor is tetrahydrobiopterin (BH4). The biochemical function of BH4 as a member of the biopteridin redox system involved in the oxidation of aromatic rings is known in the art. It plays an essential role in the biosynthesis of biologically/biochemically important compounds, such as certain amino acids (e.g. tyrosine), catecholamins and serotonin. However, the invention is not limited to BH4 as the relevant enzyme co-factor.

The enzyme to be employed according to the invention can be selected from the group consisting of phenylalanine hydroxylase, tyrosine hydroxylase, tryptophan hydroxylase, glyceryl-ether monooxygenase, endothelial nitric oxide synthase (eNOS), inducible nitric oxide synthase (iNOS), and neuronal nitric oxide synthase (nNOS). In one specific embodiment, the enzyme can be phenylalanine hydroxylase and/or tryptophan hydroxylase. The main biochemical functions of the enzymes mentioned above are also known in the art. It should be noted that the activity of any of these enzymes depends on the presence of the reducing co-factor BH4. In one embodiment phenylalanine hydroxylase or tryptophan hydroxylase are the enzymes, respectively, and BH4 is the essential co-factor.

Specifically, the following metabolite concentrations (as the relevant substrate/product ratios) are determined in order to express the enzymes activities:
the activity of phenylalanine hydroxylase is determined by measuring the phenylalanine/tyrosine ratio,
the activity of tyrosine hydroxylase is determined by measuring the tyrosine/dihydroxyphenylalanine (DOPA) ratio,
the activity of tryptophan hydroxylase is determined by measuring the tryptophan/5-hydroxytryptophan ratio (or from an analytical practical point of view the tryptophan/serotonin ratio being a surrogate),
the activity of glycerylether monooxygenase is determined by measuring the 1-alkyl-sn-glycerol/1-hydroxyalkyl-sn-glycerol ratio,
the activity of endothelial nitric oxide synthase (eNOS) is determined by measuring the arginine/citrulline ratio,
the activity of inducible nitric oxide synthase (iNOS) is determined by measuring the arginine/citrulline ratio, and
the activity of neuronal nitric oxide synthase (nNOS) is determined by measuring the arginine/citrulline ratio.

The biological sample usually is obtained from a mammal. In one embodiment a mammal can include a mouse, a rat, a guinea pig, a dog, a mini-pig, or a human. Thus, the method according to the invention is an in vitro method. For the measurement of the metabolite concentrations in the biological sample a quantitative analytical method such as chromatography, spectroscopy, and mass spectrometry is employed, while mass spectrometry can be particularly useful. The chromatography may comprise GC, LC, HPLC, and UPLC; spectroscopy may comprise UV/Vis, IR, and NMR; and mass spectrometry may comprise ESI-QqQ, ESI-QqTOF, MALDI-QqQ, MALDI-QqTOF, and MALDI-TOF-TOF. These analytical methods are generally known to the skilled person.

Potential therapeutic targets to be screened according to the method of the invention include a broad spectrum of human medical conditions such as various types of cancers, diabetes, obstructive lung disease, inflammatory bowel disease, cardiac ischemia, glomerulonephritis, macular degeneration and various neurodegenerative disorders. The method of the invention is also useful in detecting the gradual change of oxidative stress e.g. due to therapeutic effects.

Based on the above it has also been found out according to the present invention to directly use BH4 as a (bio)marker for determining the oxidative stress level in a biological sample.

Moreover the invention is also directed to a kit adapted for carrying out the method wherein the kit comprises a device which device contains one or more wells and one or more inserts impregnated with at least one internal standard. Such a device is in detail described in WO 2007/003344 and WO 2007/003343 which applications are both incorporated herein by reference.

Additionally the invention is also directed to the biomarker for determining an oxidative stress level in a biological sample itself.

The invention will be described with respect to one of the embodiments employing phenylalanine hydroxylase as follows. The underlying biochemical mechanism is described in more detail in FIG. 10. Phenylalanine hydroxylase converts phenylalanine into tyrosine in the presence of tetrahydrobiopterin (BH4). Under increased oxidative stress, i.e. the presence of more free radicals, tetrahydrobiopterin being a potent anti-oxidant itself is oxidized and thus depleted and, as a consequence thereof, is no longer present as the essential co-factor in a sufficient amount. The lack of sufficient quantities of this co-factor then causes a reduced enzymatic activity of the phenylalanine hydroxylase. Such a decreased enzymatic activity is indicated by an increased phenylalanine/tyrosine ratio and the observed accumulation of phenylalanine (in absolute terms and relative to tyrosine).

Even if the above mechanism is described herein for phenylalanine hydroxylase the present invention is not limited to measuring the activity of phenylalanine hydroxylase. Quite in contrast, similar considerations also apply with respect to other enzymes which depend on BH4 or other reducing co-factors. This will become apparent from the following examples and the claims attached.

### EXAMPLE 1

Diabetes mellitus type II is a severe metabolic disease characterized by insulin-resistance of the liver and other tissues like skeletal muscle and adipose tissue. The primary symptom is an elevated glucose concentration in peripheral blood but accompanying findings include (non-enzymatic) glycation of proteins—frequently measured as glycated hemoglobin—and increasing dysregulation of many other metabolic pathways. Due to this wide range of pathophysiological alterations, the sequelae of diabetes mellitus type II range from cardiovascular problems with end-points like myocardial infarction or stroke to nephrological and neurological diseases and represent a huge socio-economic burden which is mainly caused by the continously increasing obesity of the population in the industrial countries.

One of the most important pathomechanisms in diabetes mellitus type II is a pronounced oxidative stress which immediately causes damage to important classes of biomolecules such as proteins, lipids and nucleic acids (see above). In this example plasma samples derived from a well-established mouse model for diabetes mellitus type II were analyzed, the db/db mouse with a homozygous genetic defect in the leptin receptor pathway leading to vastly increased food uptake, obesity, and the development of insulin resistance.

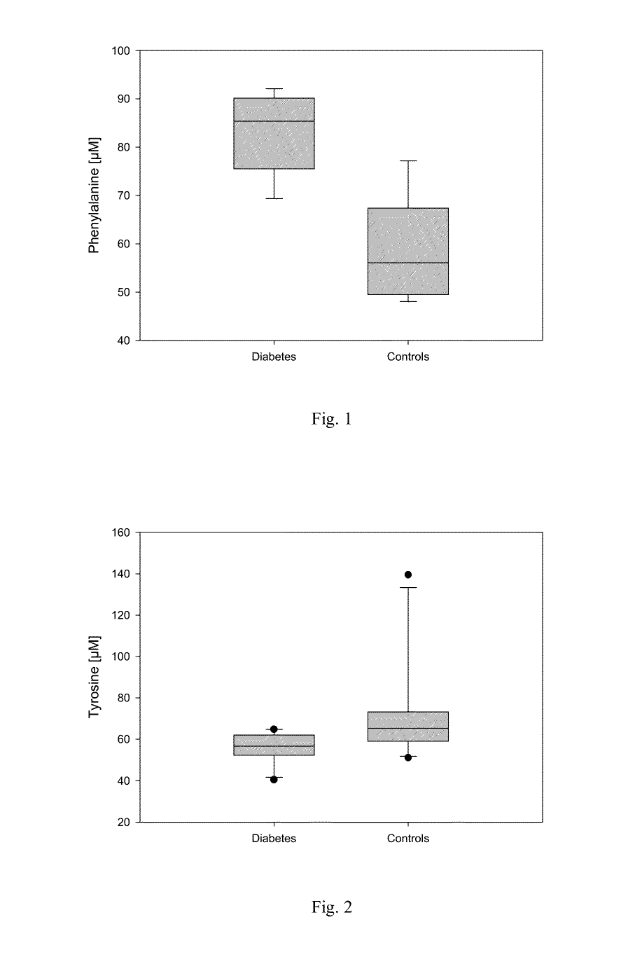

The plasma samples of the diabetic animals contained significantly elevated concentrations of the amino acid phenylalanine (82.2±8.0 μM vs. 58.9±9.7 μM; FIG. 1) while the concentrations of the amino acid tyrosine were slightly reduced (56.0±7.1 μM vs. 51.5±25.1 μM) (FIG. 2).

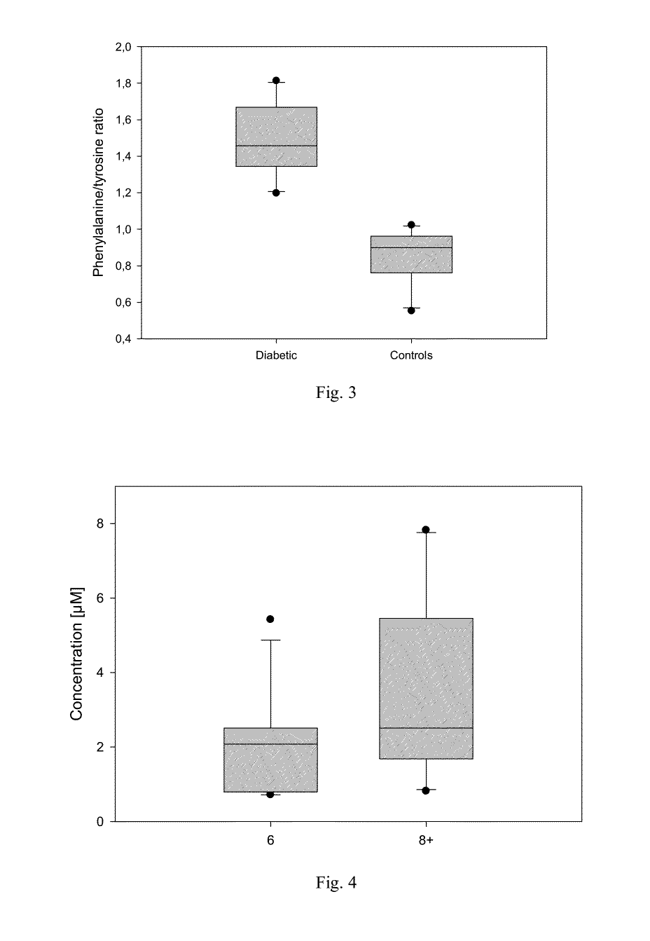

The critical ratio of phenylalanine to tyrosine concentrations indicative of the enzymatic activity of phenylalanine hydroxylase was, thus, significantly elevated in the diabetic cohort (1.48±0.19 vs. 0.86±0.14; FIG. 3).

### EXAMPLE 2

The second example is chosen from an oncological indication. Prostate cancer ranges among the most frequent causes of death in males, in males over 75 years of age it is the most frequent cancer cause of death in the US. Although the actual causal mechanisms of prostate cancer are not well understood, at least some tumors are hormone-dependent (high testosterone levels increase tumor growth in these cases), and high-fat diet is described as an additional risk factor.

Oxidative stress has often been described to play an important role in prostate cancer, either as a causative agent (Kumar et al., Cancer Res 2008; 68(6):1777-85) or as a mechanistic link between dietary factors and prostate cancer susceptibility (Fleshner & Klotz, Cancer Metastasis Rev. 1998-1999; 17(4):325-30).

In any case, oxidative stress is thought to occur in the hypoxic-ischemic core of solid tumors (Novotny et al., J Pediatr Surg. 2008 February; 43(2):330-4). This seems paradoxal at first but other conditions which involve ischemia and hypoxia (e.g. coronary infarction, stroke etc.) also induce a marked oxidative stress in the affected tissue.

In this example, metabolic biomarkers for prostate cancer in clinical samples were studied; more specifically, tissue concentrations of various classes of metabolites were compared in patients with low-grade (Gleason score 6, n=120) and high-grade (Gleason score >8, n=85) prostate cancer which significantly influences the individual prognosis after radical prostatectomy.

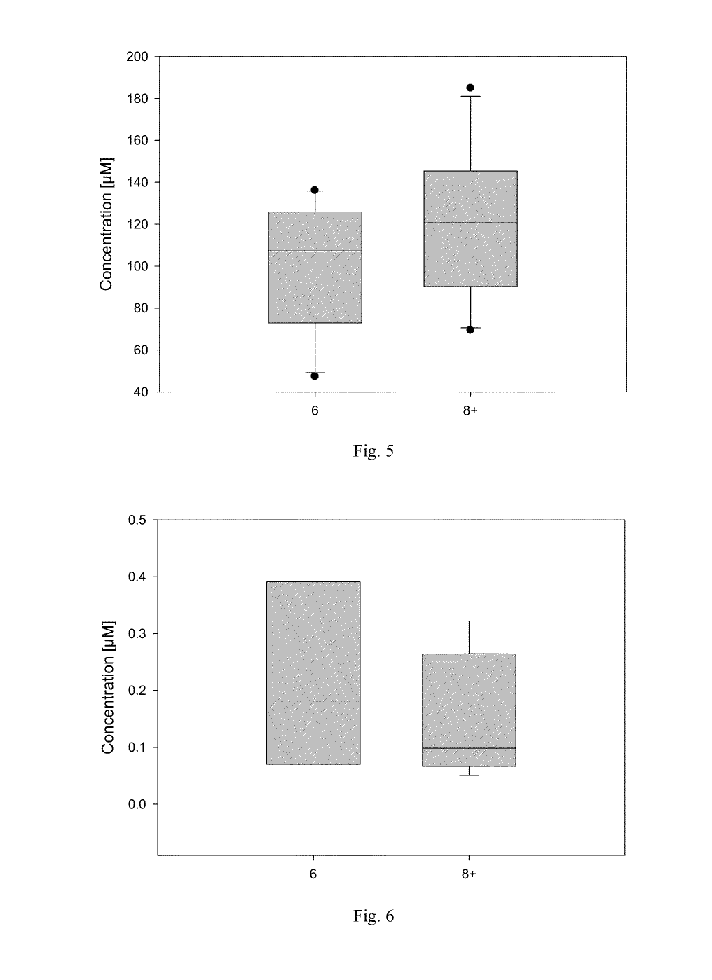

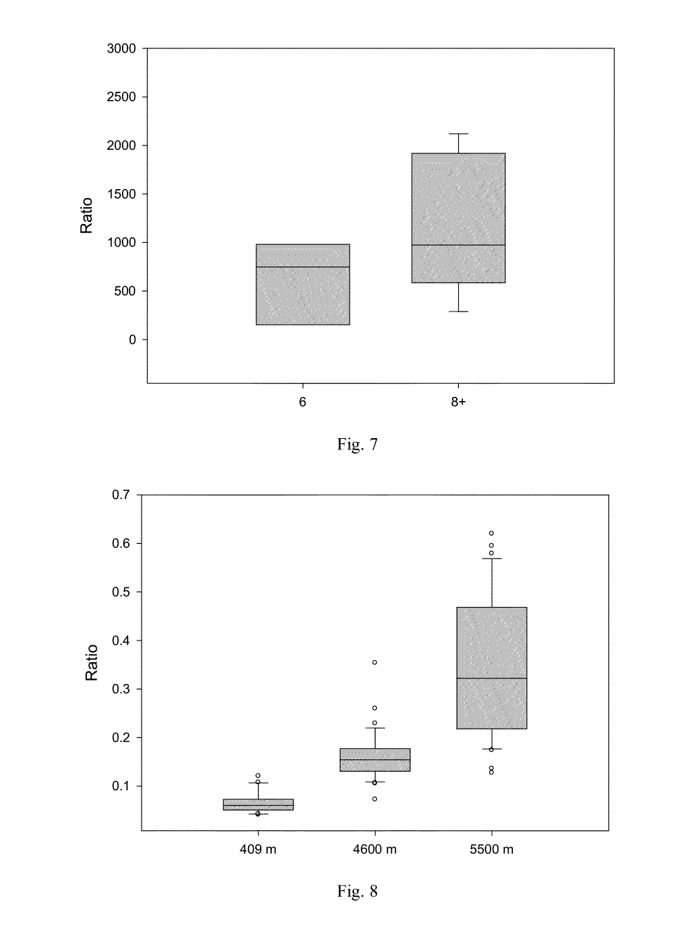

One of the main findings in this study was an elevated oxidative stress level in higher Gleason scores as demonstrated by increased concentrations of methionine sulfoxide (mean, 3.43 μM vs. 2.03 μM, FIG. 4). In the same comparison, an increase in tryptophan (mean, 119.0 μM vs. 101.3 μM, FIG. 5) and a decrease in serotonin (mean, 0.28 μM vs. 0.15 μM, FIG. 6) was identified. Moreover, the ratio of tryptophan to serotonin increased correspondingly (mean, 1169 vs. 999, FIG. 7) indicating an impaired enzymatic activity of tryptophan hydroxylase caused by depletion of tetrahydrobiopterin as an essential co-factor for this reaction.

### EXAMPLE 3

As a third example, the adaptation of 33 healthy adult human volunteers to high altitudes during an expedition on the Murtagh Ata mountain in the Pamir region and the corresponding hypoxia-related metabolic alterations was analyzed. This hypoxia is again accompanied by increasing oxidative stress as demonstrated by drastically increasing ratios of methionine sulfoxide to methionine (FIG. 8).

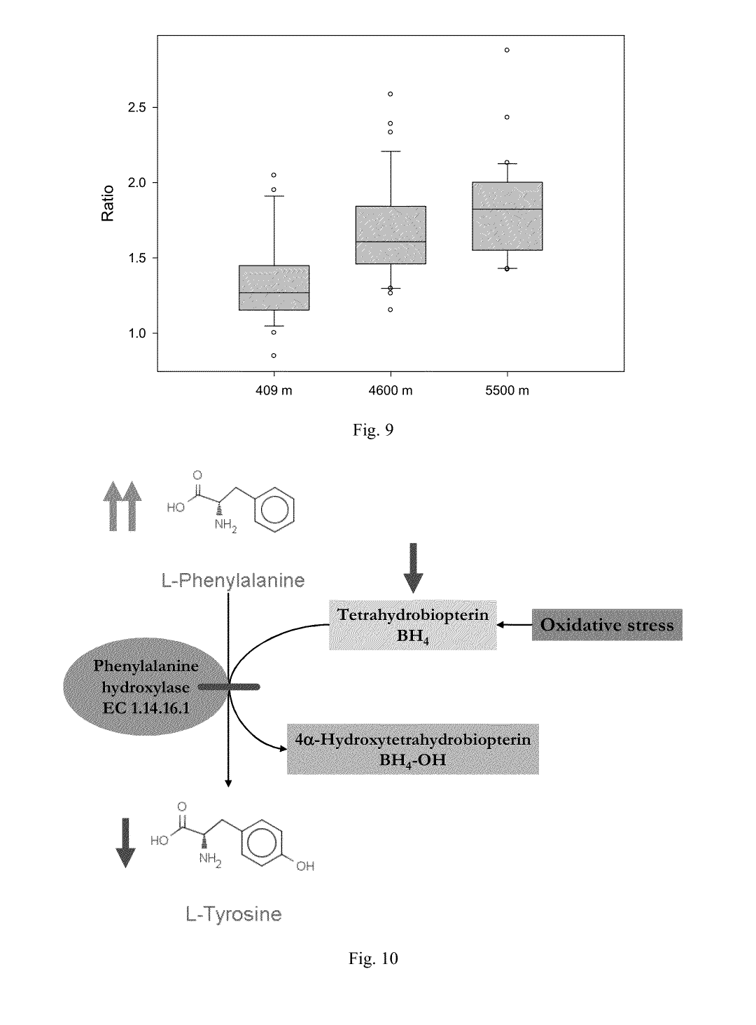

In analogy to the animal model used as the first example, an impaired activity of phenylalanine hydroxylase as measured by significantly increasing phenylalanine/tyrosine ratios in this human study could also be demonstrated (FIG. 9).

### INDUSTRIAL APPLICABILITY

The present invention provides for an improved method for determining an oxidative stress level in a biological sample which method is highly sensitive and allows for the detection of only slight changes in the oxidative stress level. The method comprises the measurement of the activity of enzymes depending on at least one reducing co-factor (e.g. BH4). Namely, an elevated level of oxidative stress, i.e. the presence of more free radicals, causes increased oxidation of certain anti-oxidative compounds which are essential co-factors for several enzymes. The lack of sufficient quantities of these co-factors then causes a reduced enzymatic activity which is measured. Thus, the decreased enzyme activity is a biomarker for an increased oxidative stress level which allows the detection of several diseases and dysfunctions in mammals.

Potential therapeutic targets to be screened according to the method of the invention include a broad spectrum of human medical conditions such as various types of cancers, diabetes, obstructive lung disease, inflammatory bowel disease, cardiac ischemia, glomerulonephritis, macular degeneration and various neurodegenerative disorders. The method of the invention is also useful in detecting the gradual change of oxidative stress e.g. due to therapeutic effects. Thus, the method and the kit for carrying out the method are highly efficient in a lot of medical fields, both in diagnosis and therapy.

1. A method for determining an oxidative stress level in a biological sample which comprises measuring the activity of one or more enzymes which depend on one or more reducing co-factors.

2. The method according to claim 1, wherein the co-factor is tetrahydrobiopterin.

3. The method according to claim 1, wherein the activity of the one or more enzymes is determined by measuring one or more metabolite concentrations.

4. The method according to claim 1, wherein the one or more enzymes is selected from the group consisting of phenylalanine hydroxylase, tyrosine hydroxylase, tryptophan hydroxylase, glyceryl-ether monooxygenase, endothelial nitric oxide synthase (eNOS), inducible nitric oxide synthase (iNOS), and neuronal nitric oxide synthase (nNOS).

5. The method according to claim 1, wherein the biological sample is obtained from a mammal, including from a mouse, a rat, a guinea pig, a dog, a mini-pig, or a human.

6. The method according to claim 1, wherein the measurement is based on a quantitative analytical method, including chromatography, spectroscopy, and mass spectrometry.

7. A kit comprising a device having one or more wells and one or more inserts impregnated with at least one internal standard, wherein the kit is adapted for carrying out the method according to claim 1.

8. A biomarker for determining an oxidative stress level in a biological sample that indicates the activity of one or more enzymes that depend on one or more reducing co-factors.

9. The biomarker according to claim 8, wherein the co-factor is tetrahydrobiopterin.

10. The biomarker according to claim 8, wherein the activity of the one or more enzymes is expressed by one or more metabolite concentrations.

11. The biomarker according to claim 8, wherein the one or more enzymes is selected from the group consisting of phenylalanine hydroxylase, tyrosine hydroxylase, tryptophan hydroxylase, glyceryl-ether monooxygenase, endothelial nitric oxide synthase (eNOS), inducible nitric oxide synthase (iNOS), and neuronal nitric oxide synthase (nNOS).

12. The method according to claim 4, wherein the one or more enzymes is phenylalanine hydroxylase and activity of the phenylalanine hydroxylase is determined by measuring the phenylalanine/tyrosine ratio.

13. The method according to claim 4, wherein the one or more enzymes is tyrosine hydroxylase and activity of tyrosine hydroxylase is determined by measuring the tyrosine/dihydroxyphenylalanine (DOPA) ratio.

14. The method according to claim 4, wherein the one or more enzymes is tryptophan hydroxylase and activity of tryptophan hydroxylase is determined by measuring the tryptophan/5-hydroxytryptophan ratio.

15. The method according to claim 4, wherein the one or more enzymes is glycerylether monooxygenase and activity of glycerylether monooxygenase is determined by measuring the 1-alkyl-sn-glycerol/1-hydroxyalkyl-sn-glycerol ratio.

16. The method according to claim 4, wherein the one or more enzymes is endothelial nitric oxide synthase and activity of endothelial nitric oxide synthase is determined by measuring the arginine/citrulline ratio.

17. The method according to claim 4, wherein the one or more enzymes is inducible nitric oxide synthase and activity of inducible nitric oxide synthase is determined by measuring the arginine/citrulline ratio.

18. The method according to claim 4, wherein the one or more enzymes is neuronal nitric oxide synthase and activity of neuronal nitric oxide synthase is determined by measuring the arginine/citrulline ratio.
